(19) United States
(12) Patent Application Publication
(10) Pub. No.: US 2007/0122419 A1
(43) Pub. Date: May 31, 2007

(54) Methods and compositions for treating atherosclerosis

(75) Inventors: Joseph L. Witztum, San Diego, CA (US); Mi-Kyung Chung, San Diego, CA (US); Gregg J. Silverman, Encinitas, CA (US); Peter X. Shaw, San Diego, CA (US); Christoph Binder, Del Mar, CA (US)

Correspondence Address: BUCHANAN, INGERSOLL & ROONEY LLP, P.O. BOX 1404, ALEXANDRIA, VA, 22313-1404, US

(73) Assignee: The Regents of the University of California, Oakland, CA (US)

(21) Appl. No.: 10/552,330

(22) PCT Filed: Apr. 12, 2004
(86) PCT No.: PCT/US04/11333

§ 371 Date: Jan. 29, 2007

Related U.S. Application Data
(60) Provisional application No. 60/462,654, filed on Apr. 11, 2003.

(51) Int. Cl. A61K 39/09; A61K 39/00; A61K 31/739

(52) U.S. Cl. 424/185.1; 514/54; 424/244.1

(57) ABSTRACT

During the progression of atherosclerosis, autoantibodies are induced to epitopes of oxidized low-density lipoprotein (OxLDL), and active immunization of hypercholesterolemic mice with OxLDL ameliorates athero-genesis. The present studies have identified anti-OXLDL autoantibodies that share complete genetic and structural identity with antibodies produced by anti-phosphorylcholine B-cell clone, T15.

### CROSS REFERENCE TO RELATED APPLICATIONS

This application claims priority from U.S. Provisional Application Ser. No. 60/462,654, filed Apr. 11, 2003, which is incorporated herein by reference.

### STATEMENTS REGARDING FEDERALLY SPONSORED RESEARCH

The invention was funded in part by Grant No. HL56989 awarded by the National Institutes of Health and by Grant No. HL69464 awarded by the National Institutes of Health. The government may have certain rights in the invention.

### TECHNICAL FIELD

The present invention relates generally to an antigenic composition useful for immunization of mammals against atherosclerosis. Further, the invention also relates to methods of producing such an antigenic composition.

### BACKGROUND

Atherosclerosis and the associated coronary artery disease and cerebral stroke represent the most common cause of death in industrialized nations. The first step in atherogenesis is the infiltration and entrapment of Low Density Lipoprotein (LDL) in the blood vessel wall. Once entrapped in the vessel wall, LDL undergoes modification through oxidation, derivatization or glycosylation. Initially, when minimally modified, endothelial cells react by secreting a chemotactic substance which attracts monocytes to the area. Monocytes then migrate through the vessel wall, transform into macrophages which then begin digesting the LDL particles as they becomes more oxidized (OxLDL). Modified LDL is cytotoxic and inhibits further migration of the macrophage out of the vessel.

During OxLDL uptake, macrophages produce cytokines and growth factors that elicit further cellular events that modulate atherogenesis such as smooth muscle cell proliferation and production of extracellular matrix. Additionally, these macrophages may activate genes involved in inflammation including inducible nitric oxide synthase.

There is now compelling evidence that both adaptive and innate immune mechanisms can modulate the progression of atherosclerosis (Binder et al., Nat Med 8:1218-1226, 2002). Among the antigens identified in atherosclerotic lesions, oxidized LDL (OxLDL) plays a prominent role (Binder et al., supra).

OxLDL contains a variety of “oxidation-specific” neoepitopes on both the lipid and protein moieties (Hörkkö et al., Free Radic Biol Med 28:1771-1779, 2000). For example, reactive decomposition products of phospholipid oxidation, such as 1-palmitoyl-2- (5-oxovaleroyl)-sn-glycero-3-phosphorylcholine (POVPC) can covalently modify protein and lipid moieties of LDL, to form adducts that retain the intact phosphorylcholine (PC) headgroup. Modification with POVPC and other decomposition products resulting from lipid peroxidation, such as malondialdehyde (MDA), leads to formation of “neo-self epitopes” that are recognized by innate and/or adaptive immunity (Palinski et al., Arteriosclerosis 10:325-335, 1990; Palinski et al., J. Clin. Invest. 98:800-814, 1996).

There are several distinct types of immunity. Nonspecific, or innate, immunity refers to the inherent resistance manifested by a species that has not been immunized (sensitized or allergized) by previous infection or vaccination. Its major cellular component is the phagocytic system, whose function is to ingest an digest invading microorganisms. Phagocytes include neutrophils and monocytes in the blood and macrophages in the tissues. Complement proteins are the major soluble component of nonspecific immunity. Acute phase reactants and cytokines, such as interferon, are also part of innate Immunity. Innate immunity relates to immunologic responses which are preprogrammed, and utilizes pattern recognition molecules to identify macromolecular structures.

Specific immunity is an immune status in which there is an altered reactivity directed solely against the antigenic determinants (infectious agent or other) that stimulated it. It is sometimes referred to as acquired immunity. It may be active and specific, as a result of naturally acquired (apparent or inapparent) infection or intentional vaccination; or it may be passive, being acquired from a transfer of antibodies from another person or animal. Specific immunity has the hallmarks of learning, adaptability, and memory. The cellular component is the lymphocyte (e.g., T-cells, B-cells, natural killer (NK) cells), and immunoglobulins are the soluble component.

Interactions of OxLDL with the innate immune system involves “pattern-recognition” scavenger receptors of macrophages, such as scavenger receptor A (SRA) and CD36, which bind oxidation-specific ligands, including PC-containing oxidized phospholipids (OXPL) and promote unregulated uptake of OXLDL (Binder et al., Nat Med 8:1218-1226, 2002). The acute phase reactant C-reactive protein (CRP), a primitive member of innate immunity and marker of atherosclerosis-related clinical events, also binds PC of OxPL of OxLDL (Chang et al., Proc Natl Acad Sci U S A 99:13043-13048, 2002).

Extensive atherosclerosis in apoE-deficient (apoE^{-/-}) mice is associated with robust antibody titers to OxLDL, enabling us to generate splenic B-cell lines from these “naïve” mice, termed “EO”, which produced monoclonal IgM autoantibodies to OxLDL (Palinski et al., J Clin Invest 98:800-814, 1996). Several different antibodies selected on the basis of binding to OxLDL were shown to recognize OxPL containing the PC headgroup, e.g. POVPC, either present as an isolated lipid, or when covalently bound to apoB. These EO antibodies did not bind to native, unoxidized phospholipids even though they contained the same PC moiety (Shaw et al., J Clin Invest 105:1731-1740, 2000). They also bound to the PC moiety of OXPL in apoptotic cells, suggesting that LDL and viable cells contain a cryptic epitope, PC, which is revealed by oxidation, or when cells undergo apoptosis. Importantly, the EO antibodies blocked the binding and degradation of OXLDL by macrophages in vitro (Hörkkö et al., J Clin Invest 103:117-128, 1999).

The genes encoding the antigen binding site of these EO antibodies (e.g. EO6) were shown to be genetically and structurally indistinguishable from antibodies produced by the previously described B-1 cell clone, T15, which is known to be specific for PC. T15 clonospecific natural antibodies confer optimal protection to mice against lethal infection with S. pneumoniae (Mi et al., Proc Natl Acad Sci U S A 97:6031-6036, 2000), in which the same PC moiety is a prominent constituent of (lipo)teichoic acid components of the cell-wall polysaccharide (C-PS) (Snapper et al., Trends Immunol 22:308-311, 2001). In most murine strains, the in vivo response to pneumococci is dominated by T15 antibodies. In vitro binding assays confirmed that the classic T15 antibody (IgA) specifically bound to OxLDL and POVPC, while the EO antibodies, such as the prototypic EO6, bound to the PC-containing antigen, C-PS. These in vitro studies suggested molecular mimicry between immunodominant PC epitopes of OxPL of OxLDL and the PC moiety of common microbial pathogens.

In summary, even though it has been known that oxidized LDL (OXLDL) will elicit an immunogenic response in a mammal, it was unknown that a similar response could be elicited to epitopes associated with OXLDL antigens derived from a pathogen.

### SUMMARY

During the progression of atherosclerosis, autoantibodies are induced to epitopes of oxidized low-density lipoprotein (OxLDL). The present study has identified anti-OxLDL autoantibodies that share genetic and structural identity with antibodies protective against common infectious pathogens. Specifically, these studies demonstrate molecular mimicry between phosphorylcholine-containing epitopes of OxLDL and the phosphorylcholine-containing epitopes found on the cell wall S. pneumoniae. Antibodies developed to oxidized phosphorylcholine-containing epitopes can be used to inhibit macrophage-mediated incorporation of oxidized lipoproteins into developing plaque and thereby inhibit the progression of atherosclerosis. Therapeutic methods which rely on such antibodies to inhibit the formation of coronary and vascular atheroma are therefore also provided. For in vitro screening, additional oxidation-specific antibodies can be identified in samples of host plasma or tissue.

In one embodiment, a method for treating or inhibiting atherogenesis by administering to a subject an immunogenic amount of a phosphorylcholine-enriched preparation. In one aspect, the phosphorylcholine-enriched preparation is derived from a component of a cell wall polysaccharide of a pathogen. The administration results in the production of antibodies that bind to a phosphorylcholine-associated epitope present in oxidized low density lipoprotein (OxLDL). The pathogen can be derived from the genus streptococcus. The cell wall component of a polysaccharide can be lipotechoic acid.

In another embodiment, a method of treating or inhibiting atherogenesis and pneumococcal infection is a subject by administering to the subject an immunogenic amount of a phosphorylcholine (PC)-enriched preparation, is provided. The administration results in the production of antibodies that bind to oxidized low density lipoprotein (OxLDL) associated with atherogenesis and to phosphorylcholine moieties associated with a cell wall polysaccharide of a pathogen. The preparation can be administered in a pharmaceutically acceptable carrier and in combination with an immunostimulant adjuvant.

In yet another embodiment, a method for ameliorating atherosclerosis in a subject by administering to the subject antibodies that bind to oxidized low density lipoprotein (OxLDL), in a pharmaceutically acceptable carrier, is provided. The antibodies can result from an immunogenic response to phosphorylcholine-containing lipoteichoic acid components of a cell wall polysaccharide of a pathogen. Such antibodies can be monoclonal or polyclonal.

The method of claim 9, wherein the pathogen can be S. pneumoniae.

In another embodiment, a method of ameliorating disease caused by atherogenesis in a subject by inducing an immune response in the subject with phosphorylcholine (PC)-enriched preparation, is provided. Generally, the subject generates antibodies that bind to phosphorylcholine associated with OxLDL, and the antibodies prevent the uptake of low density lipoproteins by macrophages, thereby ameliorating disease caused by atherogenesis. The subject can be any mammal, including a human.

In another embodiment, an anti-atherogenesis or anti-pneumococcal vaccine including an immunogenic amount of a phosphorylcholine (PC)-enriched preparation derived from a component of a cell wall polysaccharide of a pathogen, is provided. The administration results in the production of antibodies that bind to PC associated with OXLDL. The vaccine can be administered in a physiologically acceptable vehicle that further includes an immunostimulant adjuvant.

In another embodiment, an article of manufacture including packaging material and, contained within the packaging material, a pharmaceutical composition containing an immunogenic amount of a phosphorylcholine (PC)-enriched preparation, is provided. The packaging material further includes a label or package insert indicating that the composition modulates atherogenesis. In one aspect, the composition modulates atherogenesis by generating antibodies specific for low density lipoprotein. In general, the low density lipoprotein is oxidized low density lipoprotein and the phosphorylcholine (PC)-enriched preparation is derived from pneumococcus.

In another embodiment, an article of manufacture including packaging material and, contained within the packaging material, a composition containing an antibody that binds to phosphorylcholine (PC) associated with OxLDL, is provided. The packaging material further includes a label or package insert indicating that-said antibody can be used for treating atherosclerosis in a subject.

In one embodiment, an article of manufacture including packaging material and, contained within the packaging material, a vaccine that confers immunity to S. pneumoniae, is provided. The packaging material further includes a label or package insert indicating that that vaccine modulates the activity of OxLDL and can be used for treating or preventing atherogenesis in a subject.

### DESCRIPTION OF THE DRAWINGS

FIG. 1 shows that Pneumococcal immunization induces anti-OxLDL IgM antibodies.

FIG. 1J shows that anti-OxLDL IgM antibodies induced by immunization with PBS in CFA/IFA display different binding specificities.

FIG. 2 shows an ELISpot assay of frequencies of immunoglobulin secreting cells (ISC) in the spleens of the three groups of immunized mice.

FIG. 3 shows an ELISpot assay indicating the frequencies of immunoglobulin secreting cells (ISC) in the bone marrows of the three groups of immunized mice identified in FIG. 2.

FIG. 4 shows the results of Pneumococcal Intervention Study 1 for mice after 24 weeks of atherogenic diet.

FIG. 4E shows data indicating that circulating apoB-IgM immune complexes are increased in immunized mice.

FIG. 5 shows the results of Pneumococcal intervention study 2 for mice after 16 weeks of atherogenic diet.

FIG. 6 shows dilution curves of IgM, IgG1, IgG2a, and IgG3. antibody binding to C-PS in plasma of mice immunized with either pneumococcal extract (●; n=13) or PBS (o; n=15). RLU=relative light units.

FIG. 7 shows data indicating that plasma from pneumococci immunized mice inhibits OxLDL binding by macrophages.

FIG. 8 shows data indicating that PC-specific antibodies are present in human sera.

### DETAILED DESCRIPTION

Antigenic compositions for eliciting an immune response against oxidized low density protein (OxLDL) in a subject are provided. In addition, antibodies that bind to such antigens present in the composition are provided. Methods of using the compositions and antibodies to treat or inhibit atherosclerosis are also provided. Finally, articles of manufacture that contain a composition or antibody of the invention, and instructions for using them, are provided. The information provided herein identifies for the first time a structural relationship between antibodies that recognize OxLDL of athersclerotic lesions and antibodies that recognize OxLDL present in the cell walls of pathogenic organisms. The newly identified molecular mimicry provides a basis for developing vaccines to treat atherosclerosis. More particularly, these antigens, which are found at the surface of pneumococci, when formulated with an appropriate adjuvant, are used in vaccines for protection against atherosclerosis.

Unless defined otherwise, all technical and scientific terms used herein have the same meanings as commonly understood by one of ordinary skill in the art to which this invention belongs. For the purpose of clarity, some terms used in the present application will be defined below.

In the present application, the term “low density lipoprotein” (LDL) refers to the lower density form of lipoprotein present in serum. Such lipoproteins are classified according to their density into chylomicrons, very low density lipoproteins (VLDL), LDL and high density lipoproteins (HDL). They are particles comprising proteins (apolipoproteins) associated with lipids and have a micelle-like spherical structure composed of a non-polar core consisting of triacylglycerol and cholesteryl ester on one hand and apolipoproteins, cholesterol and phospholipid covering the core the other hand. Apolipoproteins and lipids are different according to each lipoprotein. More specifically, LDL has a particle size of about 180 to 280 angstroms in diameter and a density within a range of 1.006 to 1.063 (μg/ml), while its apolipoprotein is composed mainly of apoB-100. LDL is a normal blood constituent that is the body's principal means for delivery of cholesterol to tissues.

A “phosphorylcholine (PC)-enriched” preparation is any composition containing phosphorylcholine moieties that can be used to elicit an antibody response. The response generally results in antibodies that bind to the PC-containing epitopes associated with OxLDL. The term “OXLDL” refers to an oxidized form of LDL. A phosphorylcholine-enriched preparation can contain PC functionally associated with another molecule. Such molecules include oxidized phospholipids which include, but are not limited to oxidized forms of 1-palmitoyl-2-arachidonoyl-sn-glycero-3-phos- phorylcholine (Ox-PAPC), 1-palmitoyl-2-oxovaleroyl-sn-glycero-3-phosphoryl- choline (POVPC), 1-palrnitoyl-2-glutaroyl-sn-glycero-3-phosphorylcholine (PGPC),1-palmitoyl-2-epoxyisoprostane-sn-glycero-3-phosphorylcholine (PEIPC), oxidized 1-stearoyl-2-arachidonoyl-sn-glycero-3-phosphorylcholin-e (Ox-SAPC), 1-stearoyl-2-oxovaleroyl-sn-glycero-3-phosphorylcholine (SOVPC, 1-stearoyl-2-glutaroyl-sn-glycero-3-phosphorylcholine (SGPC), 1-stearoyl-2-epoxyisoprostane-sn-glycero-3-phosphorylcholine (SEIPC), 1-stearoyl-2-arachidonyl-sn-glycero-3-phosphorylethanolamine(Ox-SAPE), 1-stearoyl-2-oxovaleroyl-sn-glycero-3-phosphorylethanolamine(SOVPE), 1-stearoyl-2-glutaroyl-sn-glycero-3-phosphorylethanolamine(SGPE), 1-stearoyl-2-epoxyisoprostane-sn-glycero-3-phosphorylethanolamine(SEIPE), or related phophiolipid oxidation products and the like. It is understood that the oxidized phospholipid can be derived from any source, including eukaryotic and prokaryotic sources.

An “antigen” means a substance, such as a foreign substance, that, when introduced into the body, can stimulate an immune response. Thus, an antigenic composition is any composition comprising such an antigen, i.e. together with a suitable carrier.

As used herein, the term “vaccine” means any compound or preparation of antigens desired to stimulate a primary immune response, resulting in proliferation of the memory cells and the ability to exhibit a secondary memory or anamnestic response upon subsequent exposure to the same antigens.

The phrase “pharmaceutically acceptable” refers to molecular entities and compositions that are physiologically tolerable and do not typically produce an allergic or similar undesirable reaction, such as gastric upset, dizziness, fever and the like, when administrated to a human. Preferably, as used herein, the term “pharmaceutically acceptable” means fulfilling the guidelines and approval criteria of a European Community country's Drug Registration Agency concerning products to be used as a drug, or means that the pharmaceutically acceptable compound, composition, method or use, is listed in the European Community country's Pharmacopoeia or other generally recognised pharmacopoeia for use in animals, and more particularly in humans.

The term “pharmaceutical carrier” refers to a diluent, adjuvant, excipient, or vehicle with which the compound is administered. Such pharmaceutical carriers include but are not limited to sterile liquids, such as water and oils, including those of petroleum, oil of animal-, vegetable-, or synthetic origin, such as whale oil, sesame oil, soybean oil, mineral oil and the like. Water or aqueous solutions, saline solutions, and aqueous dextrose and glycerol solutions are preferably employed as carriers, particularly for injectable solutions, droplet-dispensed solutions and aerosols.

The term “adjuvant” refers to a compound or mixture that enhances the immune response to an antigen. An adjuvant can serve as a tissue depot that slowly releases the antigen and also as a lymphoid system activator that non-specifically enhances the immune response (Hood et al., Immunology, Second Ed., 1984, Benjamin/Cummings: Menlo Park, Calif., p. 384). Often, a primary challenge with an antigen alone, in the absence of an adjuvant, will fail to elicit a humoral or cellular immune response. Adjuvants include, but are not limited to, complete Freund's adjuvant, incomplete Freund's adjuvant, saponin, mineral gels such as aluminum hydroxide, surface active substances such as lysolecithin, pluronic polyols, polyanions, peptides, oil or hydrocarbon emulsions, keyhole limpet hemocyanins, dinitrophenol, and potentially useful human adjuvants such as BCG (bacille Calmette-Guerin) and Corynebacterium parvum. Preferably, the adjuvant is pharmaceutically acceptable.

The primary purpose of an adjuvant is to enhance the immune response to a particular antigen of interest. In the context of antibody production for research purposes, adjuvants stimulate the rapid and sustained production of high titers of antibodies with high avidity. This permits ready recovery of antibody for further research in vitro. Adjuvants have the capability of influencing titer, response duration, isotype, avidity and some properties of cell-mediated immunity.

Adjuvants may act through three basic mechanisms. The first is to enhance long term release of the antigen by functioning as a depot. Long term exposure to the antigen should increase the length of time the immune system is presented with the antigen for processing as well as the duration of the antibody response. The second is the interaction the adjuvant has with immune cells. Adjuvants may act as non-specific mediators of immune cell function by stimulating or modulating immune cells. Adjuvants may also enhance macrophage phagocytosis after binding the antigen as a particulate (a carrier/vehicle function).

Selection of an adjuvant is based upon antigen characteristics (size, net charge and the presence or absence of polar groups). Adjuvant choice is also dependent upon selection of the species to be immunized. Adjuvant selection remains largely empirical. Antigens that are easily purified or available in large quantities may be good choices for starting with the least inflammatory adjuvants for immunization. Should antibody response not be suitable, a gradual increase in the inflammatory level of the adjuvant would then be warranted. Antigens which are difficult to come by (e.g., very small quantities are available) may be better choices for complexing with the more inflammatory adjuvants such as CFA. In addition, small molecular weight compounds and others known to be weakly immunogenetic, may need to be complexed with CFA to obtain good antibody titers. Exemplary adjuvants include:

Complete Freund's Adjuvant (CFA) is a mineral oil adjuvant that uses a water-in-oil emulsion which is primarily oil. It generally contains paraffin oil, killed mycobacteria and mannide monoosleate. The paraffin oil is generally not metabolized; it is either expressed through the skin (via a granuloma or abscess) or phagocytized by macrophages.

Incomplete Freund's Adjuvant (IFA) is a mineral oil adjuvant with a composition similar to CFA but lacking killed mycobacteria.

Montanide ISA (incomplete seppic adjuvant) is mineral oil adjuvant that uses mannide oleate as the major surfactant component.

Ribi Adjuvant System (RAS) is an oil-in-water emulsion that contains detoxified endotoxin and mycobacterial cell wall components in 2% squalene.

TiterMax is a water-in-oil emulsion that combines a synthetic adjuvant and microparticulate silica with the metabolizable oil squalene. The copolymer is the immunomodulator component of the adjuvant. Antigen is bound to the copolymer and presented to the immune cells in a highly concentrated form.

Syntex Adjuvant Formulation (SAF) is a preformed oil-in-water emulsion that uses a block copolymer for a surfactant. A muramyl dipeptide derivative is the immunostimulatory component. The components are subsequently included in in squalene, a metabolizable oil.

Aluminum Salt Adjuvants are most frequently used as adjuvants for vaccine antigen delivery and are generally weaker adjuvants than emulsion adjuvants.

Nitrocellulose-adsorbed antigen provides the slow degradation of nitrocellulose paper and prolonged release of antigen.

Encapsulated or entrapped antigens permit prolonged release of antigen over time and may also include immunostimulators in preparation for prolonged release.

Immune-stimulating complexes (ISCOMs) are antigen modified saponin/cholesterol micelles. They generally form stable structures that rapidly migrate to draining lymph nodes. Both cell-mediated and humoral immune responses are achieved. Quil A and QS-21 are examples of ISCOMS.

GerbuR is an aqueous phase adjuvant and uses immunostimulators in combination with zinc proline.

Methods of stimulating an immune response in a subject against atherogenesis by administering to the subject an immunogenic amount of a phosphorylcholine (PC)-enriched preparation, wherein the administration results in the production of antibodies that bind to PC, are provided. The phosphorylcholine (PC)-enriched preparation can be administered in combination with an immunostimulant adjuvant. Further, the phosphorylcholine (PC)-enriched preparation can be derived from a bacterial source such as, for example, pneumococcus.

Anti-atherogenesis or anti-pneumococcal pharmaceutical compositions containing an immunogenic amount of a phosphorylcholine (PC)-enriched preparation derived from, for example, pneumococcus are also provided. Such compositions are useful as vaccines to prevent atherosclerotic lesions from developing. For example, such vaccines can be administered to patients prior to, contemporaneous with, or subsequent to surgical procedures performed to eliminate occluded blood vessels. The treatment would be useful to prevent or inhibit restenosis. Restenosis is a re-narrowing or blockage of an artery at the same site where treatment, such as an angioplasty or stent procedure, has already taken place. If restenosis occurs within a stent that has been placed in an artery, it is technically called “in-stent restenosis”, the end result being a narrowing in the artery caused by a build-up of substances that may eventually block the flow of blood. Compositions disclosed herein are useful for preventing restenosis by inhibiting the ability of OxLDL to interact with macrophages and promote atherogenesis.

The present invention further relates to antibodies for the prevention and/or treatment of atherosclerosis. In a first embodiment, an antibody is raised against phosphorylcholine containing OxLDL derived from a pathogen, or any functionally equivalent derivative, fragment or analogue thereof. Such antibodies are produced by administering the present an antigenic composition containing phosphorylcholine containing OxLDL derived from a pathogen as a vaccine. The antibody according to the invention is an antibody raised against oxidized LDL.

In theory, the antibodies according to the invention will be administered in one or more dosages, and the amount needed will depend on during which phase of the disease the therapy is given as well as on other factors. In order to produce such novel antibodies, the antigenic composition according to the invention will be administered to a subject in order to induce the production of the above described antibodies characteristic for atherosclerosis. Preferably, the novel antibodies will be monoclonal antibodies. Once designed, such novel antibodies may be produced by conventional techniques and used in therapy. In general, a monoclonal antibody to an epitope of the present antigen can be prepared by using a technique which provides for the production of antibody molecules from continuous cell lines in culture and methods of preparing antibodies are well known to the skilled in this field (see e.g. Coligan (1991) Current Protocols in Immunology, Wiley/Greene, NY; Harlow and Lane (1989) Antibodies: A Laboratory Manual,, Cold Spring Harbor Press, NY; and Goding (1986) Monoclonal Antibodies: Principles and Practice (2.sup.nd ed) Academic Press, New York, N.Y.). For therapeutic purposes, there may be an interest in using human antibodies. Immunization of a human host with OxLDL from a pathogen such as S. pneumoniae in one available method. Alternatively, mice or other lower mammals are immunized, and the genes encoding the variable regions of the antibodies specific for the present OxLDL from atherosclerotic tissue and OxLDL from a pathogen are isolated and manipulated by joining to an appropriate human constant region, and optionally, the complementary determining regions (CDR) used to replace the CDRs of a human antibody by genetic engineering. The resulting chimeric construct, comprising a lower variable region or CDRs and a human constant region may then be transformed into a microorganism or mammalian host cell in culture, particularly a lymphocyte, and the hybrid antibodies expressed. Also recent techniques suggest random association of immunoglobulin genes from a human host for expression in a non-human cell host e.g. prokaryotic, and screening for affinity.

For therapeutic purposes, the present antibody is formulated with conventional pharmaceutically or pharmacologically acceptable vehicles for administration, conveniently by injection. Vehicles include deionized water, saline, phosphate-buffered saline, Ringer's solution, dextrose solution, Hank's solution, etc. Other additives may include additives to provide isotonicity, buffers, preservatives, and the like. The antibody may be administered parenterally, typically intravenously or intramuscularly, as a bolus, intermittently or in a continuous regimen.

In another embodiment, pharmaceutical compositions including antigenic determinants or antibodies of the invention are provided. A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

The invention further provides an article of manufacture comprising packaging material and, contained within the packaging material, a pharmaceutical composition comprising an immunogenic amount of a phosphorylcholine (PC)-enriched preparation, wherein the packaging material comprises a label or package insert indicating that said composition modulates atherogenesis. In one aspect, antibodies specific for low density lipoprotein are included. In another aspect, the composition is an antigenic composition. The lipoprotein can be oxidized low density lipoprotein. The phosphorylcholine (PC)-enriched preparation can be derived from pneumococcus.

The invention encompasses pharmaceutical compositions comprising an antigenic composition or antibody of the invention contained in a container and labeled with instructions for use as an atherosclerosis or pneumococcus specific inhibitor. The pharmaceutical composition can be included in a kit with instructions for use of the composition in the treatment of an atherogenesis-associated disorder. The kit can further comprise instructions for using dosage. Accordingly, the invention contemplates an article of manufacture comprising packaging material and, contained within the packaging material, a composition that modulates the activity of OxLDL in atherosclerotic plaque. The packaging material includes a label or package insert indicating that the composition modulates the activity of OXLDL and can be used for treating atherosclerosis in a subject. The invention further contemplates an article of manufacture comprising packaging material and, contained within the packaging material, a compound that preferentially modulates the activity of atherosclerosis and a pathogenic infection. The packaging material includes a label or package insert indicating that the composition modulates the activity of OxLDL found in atherosclerotic plaques or OxLDL associated with the cell wall of as pathogen.

Methods for ameliorating atherosclerosis in a subject by administering to the subject a phosphorylcholine (PC)-enriched preparation, in a pharmaceutically acceptable carrier, are also provided. In addition, methods for ameliorating atherosclerosis in a subject, by administering to the subject antibodies that bind to phosphorylcholine, in a pharmaceutically acceptable carrier, are also provided. The invention further provides methods of ameliorating disease caused by atherogenesis in a subject by inducing an immune response in the subject with phosphorylcholine (PC)-enriched preparation, wherein the subject generates antibodies that bind to phosphorylcholine, and wherein the antibodies prevent the uptake of low density lipoproteins by macrophages, thereby ameliorating disease caused by atherogenesis.

### EXAMPLES

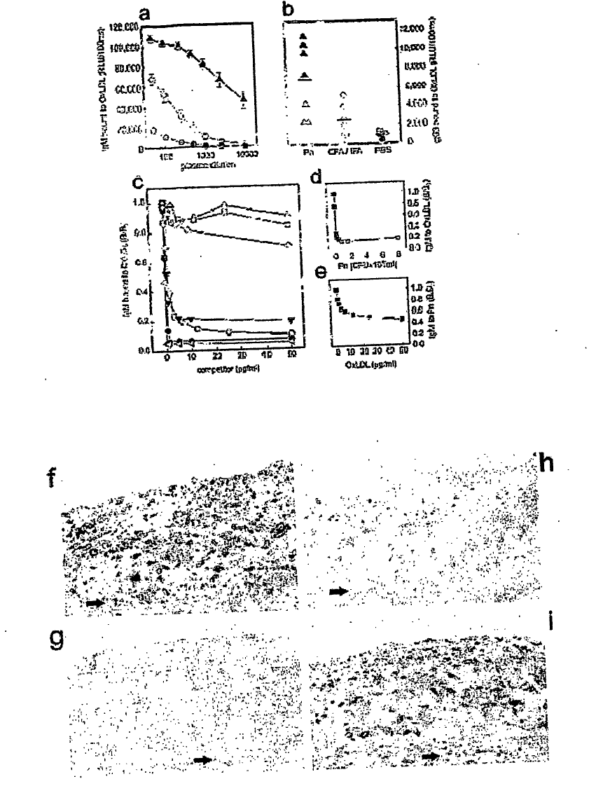

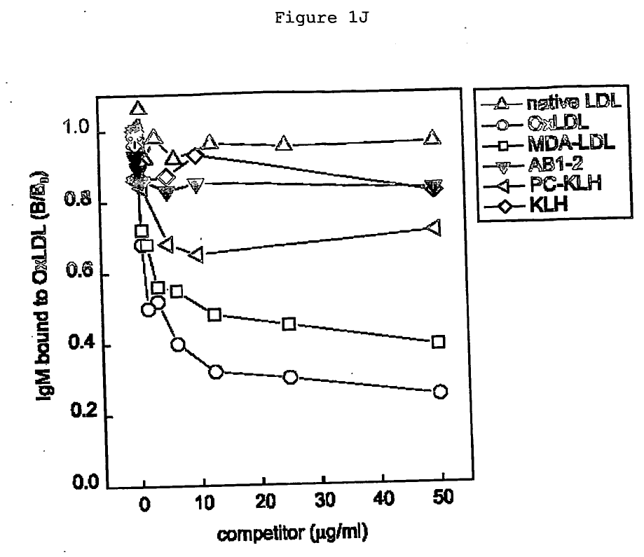

Pneumococcal immunization leads to the expansion of specific IgM antibodies to OxLDL. Immunization of chow-fed adult LDLR^{-/-} mice with pneumococcal extracts emulsified in Freund's adjuvant resulted in a strong induction of IgM titers to OxLDL, which was not observed in mice immunized with PBS alone (FIG. 1a). Induced antibodies to OxLDL were almost entirely IgM, with only weak IgG3 responses (FIG. 1b), consistent with previous reports that pneumococcal immunizations induce a thymus-independent type 2 (TI-2) response that is highly specific for PC.

Using competition immunoassay studies, it is demonstrated that IgM binding to OxLDL of pooled antisera from immunized mice was completely inhibited by OxLDL (FIG. 1c). The pneumococcal immunogen (Pn) also efficiently inhibited the binding of the antisera to OxLDL (FIG. 1d). PC, the immunodominant determinant of pneumococcal C-PS, also competed very effectively, either as a simple PC salt or as PC-KLH conjugate (FIG. 1c). Neither native (non-oxidized) LDL, MDA-LDL nor KLH (FIG. 1c), inhibited binding. The T15-clonospecific antibody, AB1-2, which identifies a determinant requiring co-expression of the canonical T15-V_{H} and T15-V_{L} region, nearly inhibited IgM binding from immune sera to OxLDL (FIG. 1c). Finally, in studies with a reciprocal design OxLDL competed up to 60% of the binding of the induced antisera to the pneumococcal immunogen (FIG. 1e). These findings confirm that IgM antibodies to OxLDL induced in vivo by pneumococcal immunization are predominantly T15-clonotypic.

Mice immunized with adjuvant alone (CFA/IFA) had a demonstrable anti-OxLDL response (FIG. 1a. However, in competition immunoassays with pooled sera from the CFA/IFA group, this binding to OXLDL was effectively competed by both OxLDL and MDA-LDL but neither PC-KLH nor AB1-2 showed a strong inhibition (FIG. 1J). Thus, the anti-OxLDL antibodies induced by immunization with CFA/IFA have different binding specificities than the predominant T15-expressing antibodies induced by pneumococcal immunization.

To further establish the molecular mimicry between epitopes of OxLDL and pneumococcal antigens, normocholesterolemic C57BL/6 mice, which do not develop atherosclerotic lesions, were immunized with OxLDL. This led to a marked increase in anti-C-PS IgM, which were predominantly T15-clonotypic as demonstrated by AB1-2.

Antisera to pneumococci recognize antigens in atherosclerotic lesions. The antisera induced by pneumococcal immunization specifically recognized epitopes in atherosclerotic lesions (Fig 1f), which was effectively abolished by pre-incubation of the antisera with excess pneumococcal immunogen (FIG. 1g). Pre-immune sera yielded no specific immunohistochemical staining (FIG. 1h). Finally, immunostaining with EO6 (FIG. 1i) resulted in a pattern closely resembling that obtained with sera from mice immunized with pneumococci.

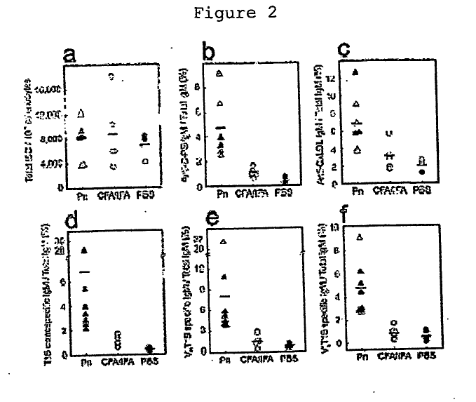

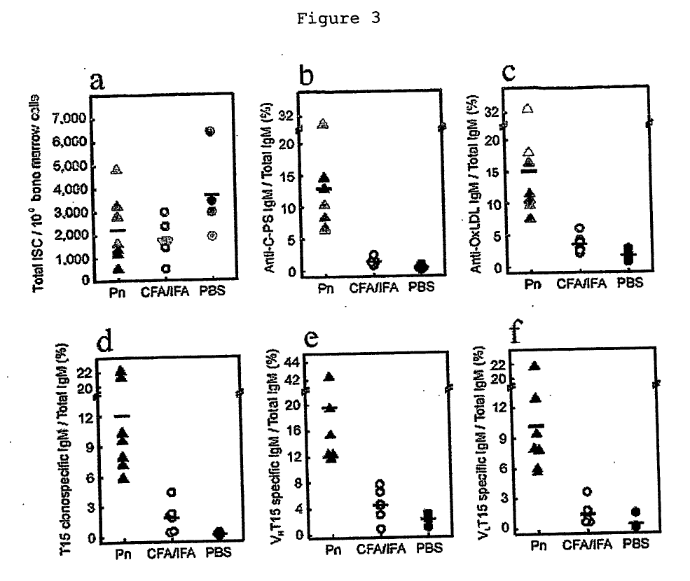

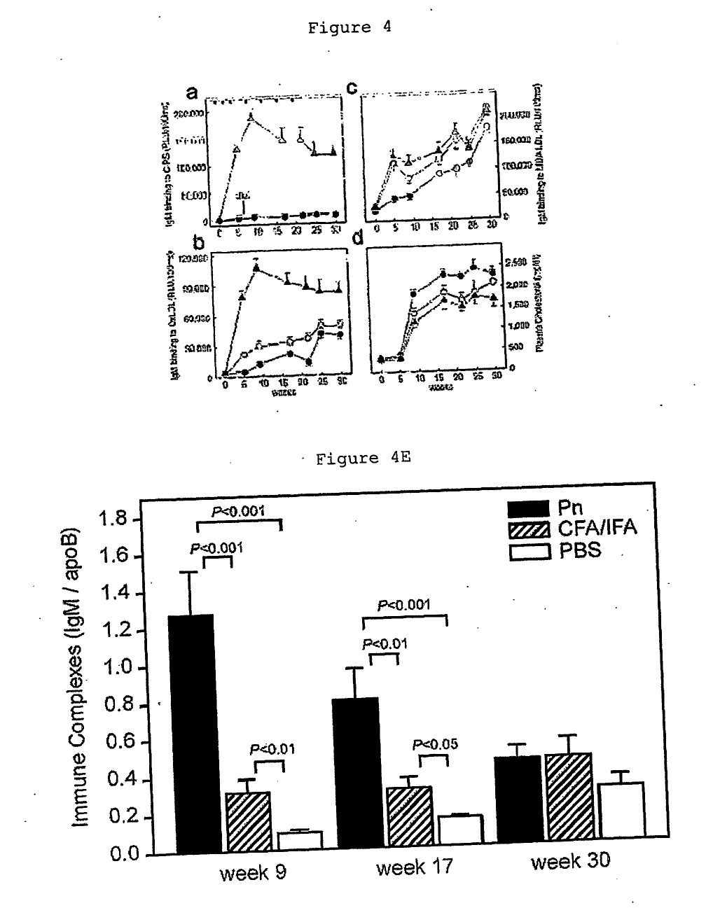

Increased frequency of cells secreting T15 idiotypic IgM in the spleens of immunized mice. To characterize the cellular origins of the induced humoral responses, the frequency and anatomic distribution of IgM-secreting cells at sacrifice, more than three months after the last immunization, were determined. In the three treatment groups, the overall frequencies of total IgM-secreting cells in the spleen did not differ significantly (FIG. 2a). Pneumococcal immunization greatly increased the frequency of cells secreting anti-pneumococcal C-PS-specific IgM (FIG. 2b), with equivalent induction in the frequency of cells secreting OxLDL-specific IgM (FIG. 2c). Furthermore, using antibody AB1-2 significantly increased frequencies of T15-clonotypic IgM-secreting cells in the spleens of mice immunized with pneumococci (FIG. 2d) is demonstrated, which was independently confirmed with two other T15-clonospecific markers, the V_{H}T15-specific antibody Tc68, and the V_{L}T15-specific antibody T139.2 (FIG. 2e and f). Equivalent patterns were also demonstrated in the bone marrows of immunized mice, but the frequencies of induced IgM-secreting cells were 50% lower than in the spleen (FIG. 3). Pneumococcal immunization reduces atherogenesis. To evaluate the effect of pneumococcal immunization on atherogenesis, LDLR^{-/-} mice were immunized with either pneumococci emulsified in Freund's adjuvant (Pn), or PBS in Freund's adjuvant (CFA/IFA), or PBS alone and then fed an atherogenic diet for 24 weeks (FIG. 4). Only the group immunized with pneumococci exhibited an IgM response specific for C-PS (FIG. 4a), which was paralleled by the induction of IgM titers to OXLDL (FIG. 4b). Controls immunized with CFA/IFA or PBS alone initially exhibited only a modest or no response to OXLDL, respectively (FIG. 4b). In contrast, IgM titers to the unrelated model epitope MDA-LDL rose initially in both groups exposed to adjuvant, independent of the cholesterol feeding, and remained slightly higher throughout the study compared to the PBS group (FIG. 4c). Only low titers of specific IgG antibodies were found in all groups. Although hypercholesterolemia per se induced a low-titered anti-OxLDL response, pneumococcal immunization led to significantly higher levels of predominantly IgM antibodies to OXLDL.

Quantification of the time-averaged plasma cholesterol exposure for each group indicated that both adjuvant groups had significantly lower plasma cholesterol than the PBS group (FIG. 4d and Table 1). However, the levels were not different between the two adjuvant groups. Triglyceride levels rose over time in all three groups, but were significantly lower only in the pneumococcal group, compared to the PBS group (P=0.02). Lipoprotein profiles by FPLC of pooled plasma from each group showed a marked reduction of the VLDL, IDL, and LDL fractions in the pneumococcal group and a similar, but lesser reduction in the CFA/IFA group.

Levels of IgM/apoB IC in the plasma of these mice were measured (FIG. 4E). Levels of IC were significantly higher in the pneumococci immunized mice during most of the study (P<0.00l). At the end of the study, however, the levels of IC in the pneumococci immunized mice decreased, despite high levels of anti-OxLDL antibodies, presumably reflecting decreased level of OXLDL in plasma at that time. Mice immunized with CFA/IFA also had higher-levels of IC when compared to the PBS group (P<0.05), which is consistent with the demonstrated increase in IgM against MDA-LDL and OXLDL.

Immunization with pneumococci significantly reduced the extent of atherosclerosis after 24 weeks of diet (Table 1). Both the percentage of aortic surface covered by Sudan IV-positive lesions in en face preparations (P<0.0l), and the area of atherosclerotic lesions in the aortic origin (P<0.05) were smaller compared to the PBS group. Surprisingly, in the CFA/IFA group atherosclerosis was decreased at the aortic origin (P<0.05), but not in the entire aorta, compared to the PBS group.

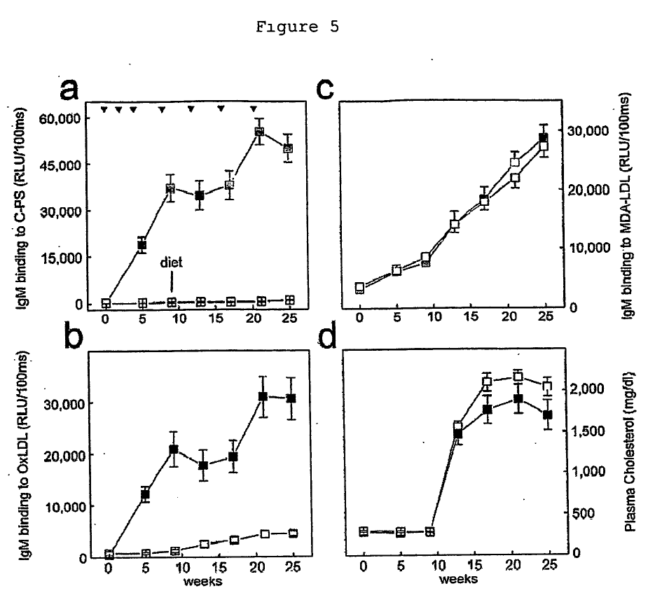

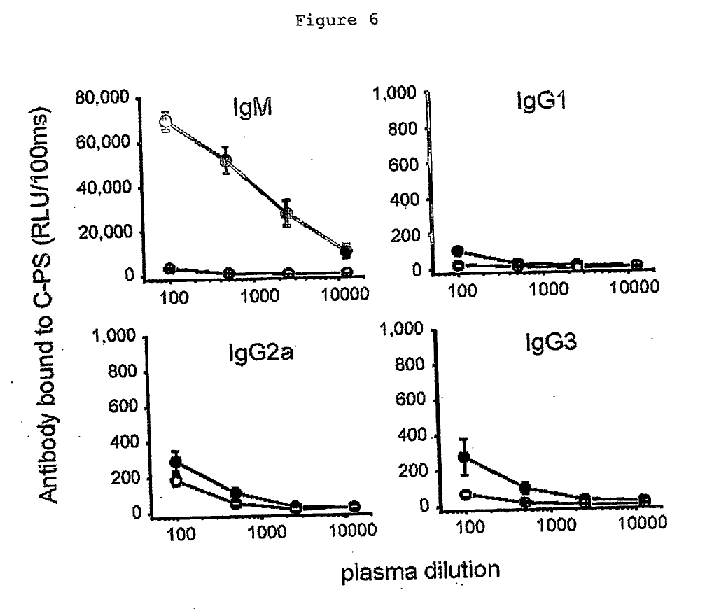

A second intervention study was initiated in which Freund's adjuvant was not used. LDLR^{-/-} mice were immunized with either pneumococci in PBS (Pn) or with PBS alone, otherwise using a similar protocol (FIG. 5a). Even without adjuvant, mice immunized with pneumococci exhibited a strong IgM response to C-PS (FIG. 5a) and to OxLDL (FIG. 5b). In contrast, the PBS group displayed only a minimal rise in anti-OxLDL IgM (FIG. 5b). Strikingly, in both groups, IgM titers to MDA-LDL rose in parallel, demonstrating that the pneumococcal immunization did not influence the development of these antibody responses (FIG. 5c). Again, pneumococcal immunization induced predominantly IgM responses, and minimal IgG titers to C-PS were induced (FIG. 6).

In the second intervention study, mice were sacrificed after 16 weeks of cholesterol-feeding. All mice gained weight equally, and time-averaged plasma cholesterol and triglyceride levels were similar (FIG. 5d and Table 1). In this setting, the increased levels of immune complexes noted in the first intervention study were not observed. Significantly, mice immunized with pneumococci had 21.5% less atherosclerosis in the aortic origin compared to the control group (0.249 mm²/section vs. 0.317 mm²/section, P<0.05) (Table 1). The extent of atherosclerosis in the entire aorta was also decreased by 8.7%, but this did not reach significance. Thus, pneumococcal immunization decreased atherosclerosis in older, more established lesions of the aortic origin. Notably, this reduction occurred despite marked hypercholesterolemia of ≈1,600 mg/dl, levels that in other models have overcome the impact of total immune deficiency (e.g. lack of T- and B-cells).

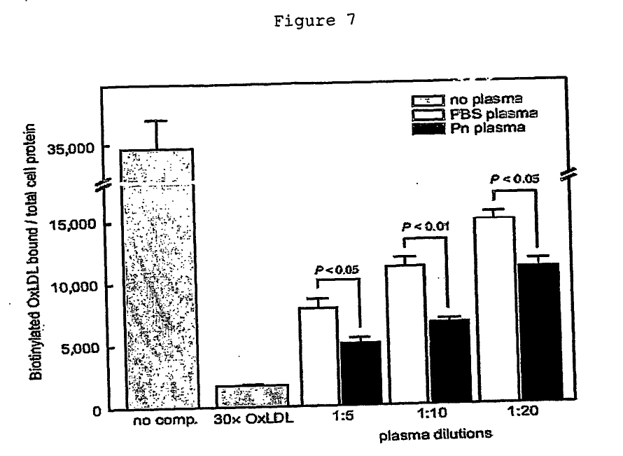

Plasma from pneumococci immunized mice block OxLDL uptake by macrophages. Monoclonal T15/EO6 IgM antibodies block the binding and uptake of OxLDL by macrophages. Therefore, the capacity of plasma from different treatment groups to inhibit the binding of OxLDL to macrophages was evaluated. Pooled plasma from mice immunized with pneumococci was considerably more effective in blocking binding of OXLDL to macrophages, compared to the plasma from control mice (FIG. 7). Similar results were seen in the first intervention study.

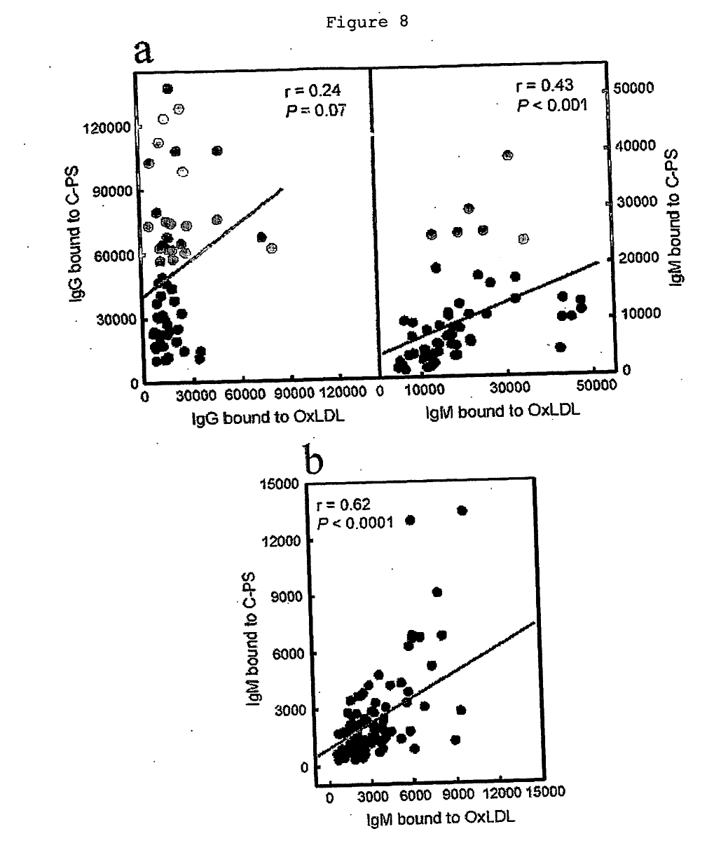

Human sera contain IgM with cross-reactivity between C-PS and OxLDL. To investigate whether epitope equivalence in the immune responses to pneumococci and OXLDL can also be observed in humans, antibody binding to C-PS and OxLDL of sera obtained from patients recently diagnosed with pneumococcal pneumonia was measured. Whereas IgG titers to the two antigens did not correlate, IgM binding showed a significant correlation (FIG. 8a). In addition, sera from hypercholesterolemic patients displayed a significant correlation between IgM titers to OxLDL IgM and C-PS (FIG. 8b). Thus, these results demonstrate the potential for similar molecular mimicry between immune responses to C-PS and OxLDL in humans.

In the current studies, immunization of cholesterol-fed LDLR^{-/-} mice with a standard pneumococcal vaccine preparation induced a high titer of T15-clonospecific OxLDL-specific IgM antibodies, which in turn reduced progression of atherosclerosis. These induced antibodies, which could be inhibited by the pneumococcal immunogen, specifically recognized determinants on OxLDL (FIG. 1), on apoptotic cells, and in atherosclerotic lesions (FIG. 1), as previously described for the “natural” T15 antibodies (i.e. those arising without immunization) (Shaw et al., J Clin Invest 105:1731-1740, 2000; Chang et al., Proc Natl Acad Sci U S A 96:6353-6358, 1999).

In a second intervention study immunization with pneumococci also induced significantly elevated antibody titers to OxLDL (FIG. 5). In contrast, the mice receiving buffer alone displayed only a very modest rise in titers to OxLDL. The fact that atherosclerosis in the aortic origin of these mice was significantly decreased in the absence of adjuvant, despite massive hypercholesterolemia, conclusively establishes a protective effect of pneumococcal immunization. The pneumococcal induced antibodies to OxLDL were almost exclusively T15 IgM (FIG. 1, 2, and FIG. 3.), demonstrating that the expansion of IgM antibodies specific for a single epitope had a significant impact on lesion formation. Thus, the present studies identify PC as a key epitope in the protective immune response to OxLDL.

Immunization with Freund's adjuvant alone induced a mild atheroprotective effect. These mice had an unexpected high titer of anti-MDA-LDL antibodies, and a modest increase in anti-OxLDL titer, which were demonstrable even before the high cholesterol diet was initiated. It is likely that immunization with this lipid containing adjuvant induced a local inflammatory reaction, which led to lipid peroxidation and the generation of MDA (and other lipid peroxidation products) that in turn generated immunogenic adducts with local proteins, including LDL. Thus, it appears likely that CFA, by several mechanisms, provides the oxidation-specific epitope MDA that serves as an immunogen. This is highly relevant as it has been shown that immunization with MDA-LDL leads to a reduction in atherogenesis (Freigang et al., Arterioscler Thromb Vasc Biol 18:1972-1982, 1998). Others have shown that Freund's adjuvant alone decreased lesion formation in apoE^{-/-} mice (Hansen et al., Atherosclerosis 158:87-94, 2001).

Evidence that T15 IgM bind to OxPL epitopes on OxLDL and prevent macrophage uptake of OxLDL suggests a mechanism that may contribute to the decreased progression of atherogenesis in the pneumococci immunized mice. Indeed, the T15 IgM enriched plasma from the pneumococcal immunized mice had an enhanced capacity to inhibit the binding of OxLDL by macrophages in vitro. Because the uptake of OxLDL via scavenger receptors (e.g. SR-A or CD36) leads to-foam-cell formation, such inhibition would be expected to impede atherogenesis. Under ordinary circumstances, IgM are primarily intravascular molecules. However, immunoglobulins, including IgM, are abundantly present in atherosclerotic lesions. It has been demonstrated that T15 antibodies are present in atherosclerotic lesions of LDLR^{-/-} mice (Shaw et al., J Clin Invest 105:1731-1740, 2000). Thus, once atherosclerotic lesions form, T15 antibodies can gain access to the subintimal space and potentially inhibit macrophage uptake of oxidized lipoproteins.

The spleen is a major anatomic source of IgM anti-OxLDL antibodies in non-immunized atherosclerotic mice, and in mice immunized with pneumococcal extracts (FIG. 2). Recently, the spleen has been recognized as the major source of natural protective anti-microbial antibodies, like those from the T15 B-cell clone (Silverman et al., J Exp Med 192:87-98, 2000; Bendelac et al., Nature Reviews Immunology 1:177-186, 2001). The spleen is also important in the maintenance of the B-1 cell pool in general and splenectomized mice do not develop anti-PC responses. The data presented herein provide a mechanistic basis to explain in part the recent observations that splenectomy of apoE^{-/-} mice enhances atherosclerosis and that this can be rescued by passive splenic B-cell transfer from apoE^{-/-} donors (Caligiuri et al., J Clin Invest 109:745-753, 2002).

The data provided herein indicate that oxidation-specific neo-self epitopes have a special relationship with recurrently arising clones that are part of a primitive tier of the host immune system, the B-1 cell pool, which has been selected during evolution for its beneficial roles in host defense and likely for protection from stressed self-strictures (e.g. OxLDL and apoptotic cells). As such, B-1 cells, typified by the T15 clone, are a major source of “natural” antibodies. In several cases, these natural antibodies have been shown to be important for initial anti-microbial (e.g. S. pneumoniae) and viral defense. The present findings demonstrate that immunizing with either OxLDL or pneumococci boosts specific anti-PC titers, which leads to a reduction in atherogenesis. These data provide direct evidence of the “housekeeping functions” for natural antibodies.

The present data indicate that PC exposure generates a “pathogen associated molecular pattern” that is recognized by pattern recognition receptors of highly conserved innate responses, that includes not only natural immunoglobulins, but scavenger receptors of macrophages, such as CD36 and SR-B-1, and CRP. The finding that immunization with pneumococcal antigen greatly increases the titer of EO6-like antibodies and markedly reduces atherogenesis directly demonstrates the possibility that exposure to pathogens, such as pneumococci, could significantly influence the course of atherogenesis.

In addition, the present studies demonstrate a correlation between IgM to OxLDL and C-PS in human subjects. The human immune response is complex and available pneumococcal vaccines have not been developed to optimize the IgM responses to C-PS (Brown et al., J Immunol 132:1323-1328, 1984). The development of IgM responses can be beneficial in the treatment of atherogenesis and pneumococcal vaccines. Thus, PC-based immunization strategies may have a therapeutic potential for ameliorating atherogenesis as well as other inflammatory diseases in which OxPL are generated.

FIG. 1 shows that Pneumococcal immunization induces anti-OxLDL IgM antibodies. (a) Dilution curve of IgM antibody binding to OxLDL in plasma of mice that received pneumococcal extract emulsified in Freund's adjuvant (Pn; filled triangles; n=7), or PBS in Freund's (CFA/IFA; open circles; n=7), or PBS alone (filled circles; n=6). RLU =relative light units. Values represent mean±SEM. (b) Titers of IgG3 antibodies to OXLDL in plasma diluted 1:50. Results are from individual mice and the horizontal bar represents the mean for the group. (c) Competition immunoassay of pooled sera for binding of plasma IgM to OxLDL with increasing concentrations of native LDL (open triangles), OxLDL (open circle), MDA-LDL (open squares), T15-clonospecific antibody AB1-2 (filled triangles), phosphorylcholine HCl (PC; filled circles), PC-KLH (left-pointing open triangles), or KLH (open diamonds). Data are expressed as a ratio of binding in the presence of competitor (B) divided by binding in the absence of competitor (B₀) and represent the mean of triplicate determinations. Results with MDA-LDL as competitor were obtained in a different experiment, using a representative plasma sample of the study. (d) Competition for binding of IgM to OxLDL by pneumococcal extract (Pn). (e) Competition for binding of IgM to pneumococcal extract (Pn) by OxLDL. (f-i) Sections of atherosclerotic aortas from balloon-catheterized, cholesterol-fed NZW rabbits were stained with antisera from pneumococci immunized mice or the monoclonal antibody EO6. Epitopes recognized are indicated by a red color; the nuclei are counterstained with methyl green. The arrow indicates the internal elastic lamina. Sections immunostained with: (f) pooled post-immune sera, (g) pooled post-immune sera pre-absorbed with excess pneumococcal antigen, (h) pooled pre-immune sera, and (i) monoclonal antibody EO6.

FIG. 1J shows that anti-OxLDL IgM antibodies induced by immunization with PBS in CFA/IFA display different binding specificities. Competition immunoassay of pooled sera for binding of plasma IgM to OxLDL with increasing concentrations of native LDL, OxLDL, MDA-LDL, T15-clonospecific antibody AB1-2, PC-KLH, or KLH. Data are expressed as a ratio of binding in the presence of competitor (B) divided by binding in the absence of competitor (B₀). These data demonstrate that the low-titered IgM anti-OxLDL antibodies induced by Freund's adjuvant are not T15 clonotypic.

FIG. 2 shows an ELISpot assay of frequencies of immunoglobulin secreting cells (ISC) in the spleens of the three groups of immunized mice. Frequencies of specific ISC in the spleens were determined by binding to wells coated with either: (a) anti-mouse-IgM antibody; (b) capsular polysaccharide (C-PS); (c) OxLDL; (d) T15-clonospecific antibody, AB1-2; (e) V_{H}-T15 specific antibody, Tc68; and (f) V_{L}-T15 specific antibody, T139.2. In panel (a), values are depicted for frequencies of total IgM-secreting cells, while values in other panels represent the number of IgM secreting cells to the indicated antigen as a percent of total IgM secreting cells. Results are from individual mice and the horizontal bar represents the mean for the group. Values were determined at time of sacrifice, more than three months after the last immunization.

FIG. 3 shows an ELISpot assay indicating the frequencies of immunoglobulin secreting cells (ISC) in the bone marrows of the three groups of immunized mice identified in FIG. 2. Mice immunized with pneumococci (Pn), mice immunized with CFA/IFA, and mice immunized with PBS alone. Frequencies of specific ISC in the bone marrows were determined by binding to wells coated with either: (a) anti-mouse-IgM antibody; (b) capsular polysaccharide (C-PS); (c) OxLDL; (d) T15-clonospecific antibody, AB1-2; (e) V_{H}-T15 specific antibody, Tc68; and (f) VL-T15 specific antibody, T139.2. In panel (a) values are depicted for frequencies of total IgM-secreting cells, while values in other panels represent the number of IgM secreting cells to the indicated antigen as a percent of total IgM secreting cells. Results are from individual mice and the horizontal bar represents the mean for the group. Values were determined at time of sacrifice, more than three months after the last immunization.

FIG. 4 shows the results of Pneumococcal Intervention Study 1 for mice after 24 weeks of atherogenic diet. (a) Time course of IgM binding to C-PS of plasma from mice immunized with pneumococci (Pn) in Freund's adjuvant (filled triangles; n=9), PBS in Freund's adjuvant (CFA/IFA; open circles; n=10) or PBS alone (filled circles; n=12). The symbols in panel a are the same for panels a-d. Six weeks after the primary immunization mice were put on an atherogenic diet (black arrow). The time points for immunizations are indicated as black arrow heads at the top of the panel. Plasma samples were obtained before the initial immunization (0 time point) and at indicated times. The final point was obtained at sacrifice. Samples were diluted 1:500 and IgM binding was determined by ELISA. Results are expressed as RLU/100 ms. (b) Binding of IgM to OxLDL. (c) Binding of IgM to MDA-LDL. (d) Time course of total plasma cholesterol levels.

FIG. 4E shows data indicating that circulating apoB-IgM immune complexes are increased in immunized mice. At week 9 and 17 of the intervention study (Study 1) the IC were significantly higher in the plasma of Pn-immunized mice compared to the other two groups. Mice immunized with CFA/IFA also exhibited significantly higher levels of immune complexes compared to the PBS group at weeks 9 and 17. At the end of the study (week 30), no significant differences were seen between the three groups. Values represent mean±SEM. Circulating immune complexes were determined by a capture assay in which LF3, a monoclonal antibody specific for murine apoB-100 1 (kindly provided by S. G. Young, Gladstone Institute of Cardiovascular Disease, San Francisco, Calif.), was coated on microtiter wells at 5 μg/ml in PBS. After washing and blocking steps, individual mouse sera, diluted 1:100 in BSA-PBS, were added to the wells and incubated for 1 hour at room temperature. After thorough washing, IgM bound to the captured apoB-containing particles was detected using an alkaline phosphatase-conjugated goat anti-mouse IgM antibody by chemiluminescent ELISA. In parallel wells, the relative amount of apoB captured in each sample was determined using biotinylated LF5, another monoclonal antibody specific for mouse apoB-100 1, followed by incubation with alkaline phosphatase-labeled NeutrAvidin and LumiPhos 530. Because LF5 binds to only one epitope of apoB-100, the amount of IgM bound to the captured LDL was then normalized for the amount of captured apoB, and expressed as a ratio of IgM counts (RLU/100ms)/apoB-100 counts (RLU/100ms) or IgM/apoB.

FIG. 5 shows the results of Pneumococcal intervention study 2 for mice after 16 weeks of atherogenic diet. (a) Time course of IgM binding to C-PS of plasma from mice immunized with pneumococci (Pn; filled squares; n=13) or PBS alone (open squares; n=15). The symbols in panel a are the same for panels a-d. Six weeks after the primary immunization mice were put on an atherogenic diet (black arrow). The time points for immunizations are indicated as black arrow heads at the top of the panel. Plasma samples were obtained before the initial immunization (0 time point) and at indicated times. The final point was obtained at sacrifice. IgM binding was determined as described in legend of FIG. 4. Note that these measurements were done with a different luminometer that gives approximately 4 times lower RLU values than the one used in the first intervention study (FIG. 4). In a separate formal analysis of antibody responses from both experiments, which assessed serial plasma dilutions utilizing the same luminometer, equivalent titers of free antibodies to OXLDL were seen in the mice receiving pneumococcal immunization in both the first and the second intervention study. (b) Binding of IgM to OXLDL. (c) Binding of IgM to MDA-LDL. (d) Time course of total plasma cholesterol levels. All values represent mean±SEM.

FIG. 6 shows dilution curves of IgM, IgG1, IgG2a, and IgG3 antibody binding to C-PS in plasma of mice immunized with either pneumococcal extract (●; n=13) or PBS (o; n=15). RLU=relative light units.

FIG. 7 shows data indicating that plasma from pneumococci immunized mice inhibits OXLDL binding by macrophages. The specific binding of biotinylated OxLDL to murine macrophages was demonstrated by incubation in the absence and presence of 30-fold excess unconjugated OxLDL (grey bars). To determine the capacity of the antisera to inhibit OxLDL binding, three dilutions of pooled plasma (1:5, 1:10, and 1:20) from mice immunized with pneumococci (Pn; black bars) or PBS (white bars) were added together with biotinylated OxLDL to macrophages. The extent of OxLDL binding is expressed RLU/100 ms per μg total cell protein.

FIG. 8 shows data indicating that PC-specific antibodies are in human sera. Correlation of (a) IgG and IgM titers to OXLDL and C-PS in sera of 29 patients before and after a recent diagnosis of pneumococcal pneumonia. (b) Correlation of IgM titers to OxLDL and C-PS in sera from hypercholesterolemic patients. Sera were diluted 1:500 and antibody binding to OxLDL or C-PS was determined by chemiluniscent ELISA. Data are expressed as relative light units per ms.

### Materials and Methods

Mice, Immunizations and diets. LDLR^{-/-} mice (10^{th} generation C57BL/6) were obtained from Jackson Laboratories (Bar Harbor, Me.).

For the initial immunization study, male and female mice, age 12-15 weeks, fed nonatherogenic chow (Harlan Teklad W860) were divided into 3 groups. Group 1 (n=7) was immunized with 10⁸ colony forming units (CFU) of pneumococci (Pn) emulsified complete Freund's adjuvant (CFA) for the primary subcutaneous immunization or incomplete Freund's adjuvant (IFA) for four subsequent intraperitoneal booster immunizations over 14-16 weeks. Group 2 (n=7) was immunized with PBS in CFA or IFA (CFA/IFA). Group 3 (n=6) received PBS alone (PBS).

For the first intervention study (with Freund's adjuvant), 36 male mice, age 15-19 weeks, were divided into 3 groups matched for body weight, age, and plasma cholesterol levels. Mice were immunized with 10⁸ CFU of Pn in CFA/IFA (Pn, n=12), or CFA/IFA alone (n=12), or PBS alone (n=12) as shown in FIG. 5a. All mice were initially fed nonatherogenic chow for 6 weeks and then an atherogenic diet, containing 21.2% milkfat and 1.25% cholesterol (TD96121, Harlan Teklad) for 24 more weeks.

During the study, four mice (2 each in the Pn and CFA/IFA group) died of anesthesia and other causes and one mouse (Pn group) was excluded from the final analysis due to lack of any weight gain.

For the second intervention study (without Freund's adjuvant), 30 male mice, age 15-16 weeks, were divided into equal groups matched for body weight, age, and plasma cholesterol. Mice in the Pn group (n=15) were injected with ⁸ bacterial CFU in 200 μl sterile PBS; PBS group (n=15) received PBS only. Mice were immunized as shown in FIG. 7. They were initially fed rodent chow for 9 weeks and then the atherogenic diet for 16 more weeks. During the study, one mouse died of anesthesia overdose and another was excluded because of the formation of extensive aneurysms in the aortic origin.

Plasma aliquots obtained at baseline and 8 days after each immunization were stored at −20° C. Plasma cholesterol and triglyceride levels were determined using an automated enzymatic assay (Boehringer Mannheim). The experimental protocol was approved by the Animal Subjects Committee of UCSD.

Immunoassays. Antibody titers were determined by chemiluminescent enzyme immunoassays, as described. To demonstrate specificity, antisera from individual Pn immunized mice (n=7) were serially diluted in PBS containing 0.27 mM EDTA and 2% BSA (BSA-PBS), to define the respective dilutions of each antiserum associated with similar binding activity to OxLDL or Pn. Equal volumes of each diluted antiserum were then pooled for the subsequent competition assay, in which increasing amounts of competitors were incubated overnight at 4° C. with a fixed and limiting dilution of the pooled antisera, and then the binding to OxLDL- or Pn-coated wells determined by chemiluminescent immunoassay.

Enzyme-linked inmunospot (ELISpot) assays. The frequencies of immunoglobulin (Ig)—and antigen—and clonospecific-secreting splenocytes and bone marrow cells were quantitated by ELISpot. Microtiter wells were coated in parallel with 5 or 10 μg/ml of either goat affinity-purified anti-mouse IgM (Jackson), OxLDL, MDA-LDL, C-PS (Statens Serum Institut, Copenhagen, Denmark), AB1-2 (a mouse IgG1) (Kearney J F, Barletta R, Quan ZS, Quintans J. Monoclonal vs. heterogeneous anti-H-8 antibodies in the analysis of the anti-phosphorylcholine response in BALB/c mice. Eur J Immunol 1981;11: 877-883), the V_{L}T15-specific antibody T139.2 , the V_{H}T15-specific antibody Tc68 (rat IgG2a) (Kenny et al., J Exp Med 176:1637-1643, 1997) or isotype controls.

Immunohistochemistry. Immunostaining of formal sucrose fixed, paraffin embedded sections of atherosclerotic lesions was performed using a 1:1,000 dilution of pre- and post-immune sera from Pn immunized mice as well as monoclonal IgM EO6 (Hörkkö et al., J Clin Invest 103:117-128, 1999). Competitive immunostaining was performed by 1 hr preincubation of the antisera with 6×10⁸ CFU of the pneumococcal extract.

Macrophage binding assay. Binding of biotinylated-OxLDL to thioglycollate-elicited peritoneal macrophages from C57BL/6 mice plated in microtiter wells was assessed by a chemiluminescent binding assay. The binding of biotinylated-OxLDL (2 μg/ml) was determined in the absence or presence of diluted pooled plasma from the immunization groups. In parallel experiments, the specificity of the binding of biotinylated-OxLDL to macrophages was determined in the absence and presence of 30-fold unconjugated OXLDL. The binding is expressed as OxLDL bound in relative light units per μg total cell protein.

Evaluation of Atherosclerosis. The extent of atherosclerosis was determined in a blinded fashion in en face preparations of the entire aortic tree, as well as in cross sections through the aortic origin, by computer-assisted image analysis as described (Tangirala et al., J Lipid Res 36:2320-2328, 1995).

Statistical Analysis. Data are presented as mean±SEM. Results were analyzed by one-way ANOVA and Student's unpaired t test.

TABLE 1
Overview of experimental groups from both intervention studies

| | | | | Atherosclerosis | |
|---|---|---|---|---|---|
| | Weight (g) | Plasma Lipids | | En Face ^{A} | Aortic Origin ^{B} |
| Groups (number) | (% gain) | TC(mg/dl) | TG(mg/dl) | (% reduction) | (% reduction) |
| Study 1 | | | | | |
| PBS (12) | 48.6 ± 1.8 (180) | 2,064 ± 127 | 773 ± 71 | 10.6 ± 0.8 | 0.630 ± 0.039 |
| CFA/IFA | 48.2 ± 1.7 (171) | 1,634 ± 150* | 622 ± 89 | 8.6 ± 1.0 (19.0) | 0.519 ± 0.025* (17.6) |
| (10) | | | | | |
| Pneumococci | 42.7 ± 2.6 (156*) | 1,446 ± 75** | 505 ± 68* | 7.2 ± 0.6** (32.0) | 0.494 ± 0.032* (21.7) |
| (9) | | | | | |
| Study 2 | | | | | |
| PBS (15) | 45.0 ± 1.3 (156) | 1,747 ± 65 | 627 ± 52 | 8.4 ± 0.6 | 0.317 ± 0.025 |
| Pneumococci | 42.5 ± 1.8 (148) | 1,532 ± 132 | 552 ± 65 | 7.7 ± 0.6 (8.7) | 0.249 ± 0.018* (21.5) |
| (13) | | | | | |

Mice were fed an atherogenic diet for 24 weeks in Study 1 and 16 weeks in Study 2, respectively. Final body weights were obtained at time of sacrifice.
TC, Total plasma cholesterol;
TG, triglycerides (measured as the area under the cholesterol -curve over time divided by days of cholesterol feeding).
^{A} En Face measurements are given in percent lesions of the aorta;
^{B} atherosclerosis in the aortic origin was analyzed by cross sections through the aortic origin and values represent the average mm²/section; percent reduction in comparison to the respective PBS group;
asterisks indicate values that are statistically different from the respective PBS group (**P < 0.01, and *P < 0.05).
Data are mean ± SEM

What is claimed is:

1. A method of treating or inhibiting atherogenesis in a subject, the method comprising administering to the subject an immunogenic amount of a phosphorylcholine (PC)-enriched preparation derived from a component of a cell wall polysaccharide of a pathogen, wherein the administration results in the production of antibodies that bind to oxidized low density lipoprotein (OxLDL).

2. The method of claim 1, wherein the phosphorylcholine (PC)-enriched preparation is administered in combination with an immunostimulant adjuvant.

3. The method of claim 1, wherein the pathogen is streptococcus.

4. The method of claim 3, wherein the streptococcus is S. pneumoniae.

5. The method of claim 1, wherein the cell wall component of a polysaccharide is lipotechoic acid.

6. A method of treating or inhibiting atherogenesis and pneumococcal infection in a subject, the method comprising administering to the subject an immunogenic amount of a phosphorylcholine (PC)-enriched preparation, wherein the administration results in the production of antibodies that bind to oxidized low density lipoprotein (OxLDL) associated with atherogenesis and to phosphorylcholine moieties associated with a cell wall polysaccharide of a pathogen.

7. A method for ameliorating atherosclerosis in a subject, the method comprising administering to the subject a phosphorylcholine (PC)-enriched preparation, in a pharmaceutically acceptable carrier, wherein the phosphorylcholine (PC)-enriched preparation is derived from OxLDL or phosphorylcholine associated with the cell wall of S. pneumoniae.

8. The method of claim 7, wherein the phosphorylcholine (PC)-enriched preparation is administered in combination with an immunostimulant adjuvant.

9. A method for ameliorating atherosclerosis in a subject, the method comprising administering to the subject antibodies that bind to oxidized low density lipoprotein (OxLDL), in a pharmaceutically acceptable carrier, wherein the antibodies result from an immunogenic response to lipoteichoic acid components of a cell wall polysaccharide of a pathogen.

10. The method of claim 9, wherein the antibody is monoclonal or polyclonal.

11. The method of claim 9, wherein the pathogen is streptococcus.

12. The method of claim 11, wherein the streptococcus is S. pneumoniae.

13. A method of ameliorating disease caused by atherogenesis in a subject, the method comprising:
inducing an immune response in the subject with phosphorylcholine (PC)-enriched preparation, wherein the subject generates antibodies that bind to phosphorylcholine associated with OXLDL, and wherein said antibodies prevent the uptake of low density lipoproteins by macrophages, thereby ameliorating disease caused by atherogenesis.

14. The method of claim 13, wherein the subject is human.

15. An anti-atherogenesis or anti-pneumococcal vaccine comprising an immunogenic amount of a phosphorylcholine (PC)-enriched preparation derived from a component of a cell wall polysaccharide of a pathogen, wherein the administration results in the production of antibodies that bind to PC associated with OXLDL, and a physiologically acceptable vaccine vehicle.

16. The vaccine of claim 15, wherein said vehicle comprises an effective amount of an immunostimulant adjuvant.

17. The vaccine of claim 15, wherein the pathogen is Streptococcus.

18. An article of manufacture comprising packaging material and, contained within the packaging material, a pharmaceutical composition comprising an immunogenic amount of a phosphorylcholine (PC)-enriched preparation, wherein the packaging material comprises a label or package insert indicating that said composition modulates atherogenesis.

19. The article of claim 18, wherein the composition modulates atherogenesis by generating antibodies specific for low density lipoprotein.

20. The article of claim 19, wherein the low density lipoprotein is oxidized low density lipoprotein.

21. The article of claim 7, wherein the phosphorylcholine (PC)-enriched preparation is derived from pneumococcus.

22. An article of manufacture comprising packaging material and, contained within the packaging material, a composition comprising an antibody that binds to phosphorylcholine (PC) associated with OXLDL, wherein the packaging material comprises a label or package insert indicating that said antibody can be used for treating atherosclerosis in a subject.

23. The article of claim 22, wherein the antibody is generated from a phosphorylcholine (PC)-enriched preparation derived from S. pneumoniae.

24. An article of manufacture comprising packaging material and, contained within the packaging material, a vaccine that confers immunity to S. pneumoniae, wherein the packaging material comprises a label or package insert indicating that said vaccine modulates the activity of OxLDL and can be used for treating or preventing atherogenesis in a subject.

25. An article of manufacture comprising packaging material and, contained within the packaging material, an antibody that preferentially binds to S. pneumoniae, wherein the packaging material comprises a label or package insert indicating that said antibody can be used for treating a subject having an arteriosclerosis-associated disorder.

26. A method for treating or inhibiting atherogenesis in a subject, the method comprising administering to the subject an immunogenic amount of a phosphorylcholine-enriched preparation, wherein the administration results in the production of antibodies that bind to a phosphorylcholine-associated epitope present in oxidized low density lipoprotein (OxLDL).

27. The method of claim 26, wherein the phosphorylcholine-enriched preparation is derived from a phospholipid.

28. The method of claim 27, wherein the phospholipid is selected from the group consisting of oxidized forms of 1-palmitoyl-2-arachidonoyl-sn-glycero-3-phos- phorylcholine (Ox-PAPC), 1-palmitoyl-2-oxovaleroyl-sn-glycero-3-phosphoryl-choline (POVPC), 1-palrnitoyl-2-glutaroyl-sn-glycero-3-phosphorylcholine (PGPC), 1-palmitoyl-2-epoxyisoprostane-sn-glycero-3-phosphorylcholine (PEIPC), oxidized 1-stearoyl-2-arachidonoyl-sn-glycero-3-phosphorylcholin-e (Ox-SAPC), 1-stearoyl-2-oxovaleroyl-sn-glycero-3-phosphorylcholine (SOVPC, 1-stearoyl-2-glutaroyl-sn-glycero-3-phosphorylcholine (SGPC), 1-stearoyl-2-epoxyisoprostane-sn-glycero-3-phosphorylcholine (SEIPC), 1-stearoyl-2-arachidonyl-sn-glycero-3-phosphorylethanolamine(Ox-SAPE), 1-stearoyl-2-oxovaleroyl-sn-glycero-3-phosphorylethanolamine(SOVPE), 1-stearoyl-2-glutaroyl-sn-glycero-3-phosphorylethanolamine (SGPE), and 1-stearoyl-2-epoxyisoprostane-sn-glycero-3-phosphorylethanolamine (SEIPE).

29. The method of claim 27, wherein the phospholipid is derived from a cell wall of a pathogen.

30. The method of claim 29, wherein the pathogen is derived from the genus streptococcus.

31. The method of claim 30, wherein the streptococcus is S. pneumoniae.

32. The method of claim 29, wherein the cell wall component of a polysaccharide is lipotechoic acid.
